(19) United States
(12) Patent Application Publication
(10) Pub. No.: US 2017/0128478 A1
(43) Pub. Date: May 11, 2017

(54) Skin Treatment Lotion

(71) Applicant: Jerold Michaelson, Dallas, TX (US)

(72) Inventor: Jerold Michaelson, Dallas, TX (US)

(21) Appl. No.: 14/937,779

(22) Filed: Nov. 10, 2015

(51) Int. Cl. A61K 31/7048; A61K 47/10; A61K 47/46; A61K 47/02

(52) U.S. Cl. CPC A61K 31/7048; A61K 47/02; A61K 47/10; A61K 47/46

(57) ABSTRACT

A system and method for a skin treatment lotion. The lotion has a protecting agent, a moisture absorber, a binding agent, water, and an antibiotic agent or hydrocortisone. In one example, the protecting agent is zinc oxide, the moisture absorber is talcum, the binding agent is glycerin, and the antibiotic agent comprises neomycin. The lotion is applied topically to treat skin irritants. In one example, the lotion is stored and dispensed in a dispenser. This allows for controlled dispensing of the lotion as well as preventing the water within the lotion from evaporating.

### BACKGROUND OF THE INVENTION

Technical Field

The present invention relates to a system and method for treating skin.

Description of Related Art

Skin irritation can take many forms. These include ant bites, blisters, cuts, sores, etc. Treating skin irritation is often difficult as many treatments can further irritate the skin.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 is a flow chart of the method of making the lotion in one embodiment.

### DETAILED DESCRIPTION

Several embodiments of Applicant's invention will now be described with reference to the drawings. Unless otherwise noted, like elements will be identified by identical numbers throughout all figures. The invention illustratively disclosed herein suitably may be practiced in the absence of any element which is not specifically disclosed herein.

A skin treatment lotion, and a method of making the same, is provided which can be used to treat skin. A skin treatment lotion, as used herein, refers to a fluid which is applied topically to treat the skin. The skin treatment lotion can be used to treat a variety of irritants. As used herein, a skin irritant includes any irritation or malady located on the skin which causes pain, itching or irritation. These include, but are not limited to, bug bites such as ant bites, blisters, sores, cuts, scrapes, irritants due to contact with plants, etc.

In one embodiment the skin treatment lotion comprises a white pourable liquid. The lotion clots up over time covering the irritant so that it may heal. In clotting up, the lotion hardens to create a solid protective barrier. In one embodiment the lotion clots up in a matter of a few minutes.

TABLE 1
Formulation of Skin Treatment Lotion

| Ingredient | Amount | |
|---|---|---|
| Protecting Agent | 10 | grams |
| Moisture Absorber | 10 | grams |
| Binding Agent | 7.5 | cc |
| Water | Sufficient to make 60 | cc |
| Antibiotic Agent | 0.15 | grams |

As noted above, in one embodiment the skin treatment lotion comprises a protecting agent, a moisture absorber, a binding agent, water, and an antibiotic agent. Each of these ingredients will be discussed in more detail below.

The specific formulation for the skin treatment lotion in one embodiment is disclosed in Table 1, below. This formulation is for illustrative purposes only and should not be deemed limiting.

TABLE 2
Formulation of Skin Treatment Lotion

| Ingredient | Amount | |
|---|---|---|
| Zinc Oxide | 10 | grams |
| Talcum | 10 | grams |
| Glycerin | 7.5 | cc |
| Water | Sufficient to make 60 | cc |
| ¼% Neomycin | 0.15 | grams |

The above formulation of skin treatment lotion results in approximately 60 mL of lotion which weighs approximately 75.84 grams. Accordingly, one embodiment utilizing the above formulation has a density of about 12.64 grams per milliliter. In one embodiment 60 mL of lotion weights between about 70 grams and 80 grams. In one embodiment the lotion comprises no alcohol so it does not result in the “sting” associated with lotions which comprise alcohol. Further, as the lotion coats the irritant it stops the pain and aids in the healing process.

The method of making the above formulation will be discussed below. First, each of the ingredients discussed above will be addressed.

As noted, the skin treatment lotion in one embodiment comprises a binding agent. The binding agent, among other benefits, suspends and binds the ingredients in the lotion. In one embodiment the binding agent comprises glycerin. Glycerin is hygroscopic, meaning it easily absorbs water from the air. However, when glycerin is delivered with a water solution, glycerin acts as a humectant. This means the glycerin actually attracts moisture to the skin. Such a property is helpful in a lotion in that it offers the lotion a moisturizing quality, drawing moisture up through the skin and slowing or preventing excessive drying or dehydration. Further, such a property aids in the healing of the irritant. The glycerin further keeps the ingredients in suspension.

In one embodiment the glycerin is added in liquid form. The amount of glycerin in the final lotion can vary upon application. In the embodiment discussed above, a 60 cc lotion will comprise about 7.5 cc of glycerin which is about 12.5% by volume. In other embodiments the glycerin comprises between about 5% to about 25% of the final lotion formulation. In still other embodiments, the lotion does not comprise glycerin.

Aside from glycerin, there are several other binding agents which can be utilized. These include, but are not limited to, almond oil, coconut oil, palm oil, maple syrup, and honey. One or more of these binding agents can require a dispersant to help distribute the binding agent throughout the solution.

As noted, the skin treatment lotion described in Table 1 further comprises a protecting agent. A protecting agent is an ingredient which helps form a protective coating over the irritant. In one embodiment the protecting agent comprises zinc oxide. Zinc oxide is a white powder which is generally insoluble in water. Zinc oxide is a mild astringent with some antiseptic properties. In one embodiment the zinc oxide acts as a skin protectant used to protect and treat the irritants discussed herein. In one embodiment the zinc oxide forms a barrier on top of the skin which protects the area from moisture and other irritants. This barrier helps allow the irritant to heal. Zinc oxide is available in various formulations including cream, ointment, powder, and spray solution. Either of these delivery mechanisms can be used to deliver zinc oxide to the skin treatment lotion.

In one embodiment the zinc oxide is added in powder form. The amount of zinc oxide in the final lotion can vary depending upon the application. In one embodiment discussed above about 10 grams of zinc oxide is added for a 60 cc dosage of lotion. Accordingly, in one embodiment the lotion comprises about 13% by weight zinc oxide. In other embodiments the lotion comprises between about 5% and about 25% by weight zinc oxide.

While one embodiment utilizing zinc oxide has been discussed, this is for illustrative purposes and should not be deemed limiting. In other embodiments other ingredients can be substituted for the zinc oxide. These include but are not limited to talcum powder, and calamine.

As noted, the skin treatment lotion in Table 1 further comprises a moisture absorber. A moisture absorber helps absorb any excess moisture and helps the irritant heal. In one embodiment the moisture absorber comprises talcum. In one embodiment, talcum is made from finely ground talc, an extremely soft mineral. Talcum can take many forms, but in one embodiment talcum powder is utilized.

Talcum acts as a moisture absorber. By sucking up moisture from the surrounding area, talcum keeps the skin dry. This can reduce the risk of rashes and chafing. It also aids in the healing process.

In one embodiment the talcum is added in powder form. The amount of talcum in the final lotion can vary depending upon the application. In one embodiment discussed above about 10 grams of talcum is added for a 60 cc dosage of lotion. Accordingly, in one embodiment the lotion comprises about 13% by weight talcum. In other embodiments the lotion comprises between about 5% and about 25% by weight talcum. While one embodiment comprising talcum has been discussed, in other embodiments calamine can be substituted for talcum.

In one embodiment, the formulation further comprises an antibiotic. The antibiotic can comprise virtually any topical antibiotic known in the art. These include, but are not limited to, neomycin, clindamycin, and erythromycin. Antibiotics are a type of antimicrobial used in the treatment and prevention of bacterial infection. Additionally, antibiotics can kill or inhibit the growth of bacteria. Many antibiotics are also effective against fungi and protozoans. The presence of an antibiotic helps heal the skin irritant as well as prevent a localized infection.

As noted, in one embodiment, the antibiotic comprises neomycin. Neomycin is an aminoglycoside antibiotic which can be applied topically via a cream, ointment, liquid form, etc. Neomycin helps prevent or treat skin infections caused by bacteria.

Various strengths of neomycin can be utilized. In one embodiment ¼% Neomycin in used whereas in other embodiments ⅛% neomycin is used. in one embodiment, to make a 60 mL lotion, about 0.15 grams are utilized.

In one embodiment, the lotion further comprises water. The water can comprise typical tap water, but in one embodiment the water comprises de-ionized water. The water serves as the solute in which the various ingredients can be dispersed. The water is the delivery mechanism which delivers the various ingredients to the skin irritant. In one embodiment water is added last and is added to achieve a desired final volume of the lotion. Thus, for example, if 60 cc of the lotion is desired, the water would be added as necessary until the volume of 60 cc was obtained. In one embodiment the user adds a quantity sufficient to make the desired volume, also referred to as QSAD.

As noted, while various ingredients have been discussed above, this is for illustrative purposes only and should not be deemed limiting. Various ingredients can be substituted, added, or replaced depending on the specific treatment desired. As but one example, in one embodiment for treating poison ivy, the antibiotic is replaced with 1% hydrocortisone. Hydrocortisone is a steroid which prevents inflammation.

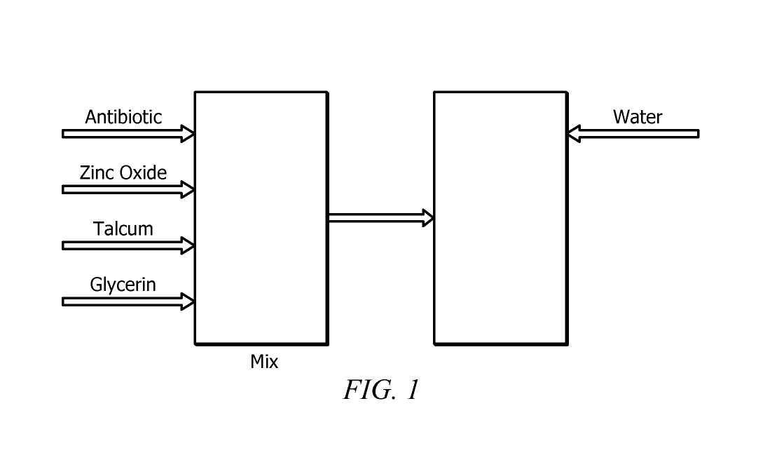

FIG. 1 is a flow chart of the method of making the lotion in one embodiment. On the left side of the figure, all the non-water ingredients, such as antibiotics, zinc oxide, talcum, and glycerin are added on the left. Thereafter, water is added in a sufficient quantity to make the desired volume. The ingredients must then be properly mixed for a sufficient time to achieve an approximately uniform solution. The resulting mixture is the lotion. In one embodiment, no external heat is necessary during mixing.

After the lotion is prepared, the lotion is transferred to a dispenser. A dispenser, as used herein, refers to an item which can store and dispense an amount of liquid. The dispenser can comprise a receptacle with a cap. In one embodiment the dispenser comprises a dispenser base which stores the lotion and a cap which seals the dispenser base. The dispenser base can be rigid or it can be squeezable. The dispenser can comprise virtually any cross-sectional shape known in the art including round, oval, square, etc.

In one embodiment the dispenser comprises a top with a narrow opening. In one embodiment the dispenser comprises a body which has a body diameter, and an opening which has an opening diameter. In one embodiment the body diameter is greater than the opening diameter. In one embodiment the body diameter is greater than five times greater than the opening diameter. In one embodiment the opening diameter comprises a dropper. This allows drops of lotion to be applied.

A reduced diameter opening offers several benefits. First, this allows for easier application of the lotion. The dispenser can be squeezed when inverted, and the reduced diameter opening will only allow drops of the desired size to be dispersed from the bottle. In such embodiments the dispenser and the dispenser opening function as a dropper. This has the benefit in allowing controlled and metered application of the lotion to the skin irritant.

A second advantage of the reduced diameter opening is increased shelf-life of the lotion. In one formulation of the lotion, the lotion can dry out over time. This is believed to be due to the evaporation of water within the lotion. As the water evaporates, the various ingredients are no longer suspended in the solution and instead precipitate out in solid form. The dehydration can be reduced by minimizing the exposure of the lotion to air. One way to accomplish this is by having a reduced diameter opening, such as a dropper. This minimizes contact of the lotion with the air, and consequently, reduces evaporation and increases shelf-life.

Once the lotion is in the dispenser, it can be dispensed from the dispenser as necessary. In one embodiment lotion is dispensed from the dispenser and applied topically to the skin irritant. An effective application coats the irritant in a thin layer of lotion which quickly dries. The lotion treats the irritant and reduces pain and irritation. Further, the thin layer of dried lotion protects the irritant.

While the invention has been particularly shown and described with reference to a preferred embodiment, it will be understood by those skilled in the art that various changes in form and detail may be made therein without departing from the spirit and scope of the invention.

### ADDITIONAL DESCRIPTION

The following clauses are offered as further description of the disclosed invention.
Clause 1. A lotion for treating an irritant, said lotion comprising:
   a protecting agent;
   a moisture absorber;
   a binding agent;
   water;
   an antibiotic agent.
Clause 2. The lotion of any proceeding or preceding clause wherein said protecting agent comprises zinc oxide, wherein said moisture absorber comprises talcum, wherein said binding agent comprises glycerin, and wherein said antibiotic agent comprises neomycin.
Clause 3. The lotion of any proceeding or preceding clause comprising about 10 grams of zinc oxide, about 10 grams of talcum, about 7.5 cc of glycerin, and neomycin.
Clause 4. The lotion of any proceeding or preceding clause wherein water is added to a quantity sufficient to make 60 milliliters.
Clause 5. The lotion of any proceeding or preceding clause comprising a density of about 12.64 g/mL.
Clause 6. The lotion of any proceeding or preceding clause wherein said lotion comprises about 5% to about 25% by weight zinc oxide, further wherein the lotion comprises between about 5% and about 25% by weight talcum,
Clause 7. The lotion of any proceeding or preceding clause wherein said lotion does not comprise alcohol.
Clause 8. A system for treating skin, said system comprising:
   a dispenser for dispensing lotion, said dispenser having a body and a top, wherein said body has a diameter greater than the diameter of said top;
   a lotion located in said dispenser, said lotion comprising:
      a protecting agent;
      a moisture absorber;
      a binding agent;
      water;
      an antibiotic agent.
Clause 9. The system of any proceeding or preceding clause wherein said protecting agent comprises zinc oxide, wherein said moisture absorber comprises talcum, wherein said binding agent comprises glycerin, and wherein said antibiotic agent comprises neomycin.
Clause 10. The system of any proceeding or preceding clause comprising about 10 grams of zinc oxide, about 10 grams of talcum, about 7.5 cc of glycerin, and neomycin.
Clause 11. The system of any proceeding or preceding clause wherein said dispenser comprises a dropper.
Clause 12. The system of any proceeding or preceding clause wherein said diameter of said body is more than 5 times the diameter at the top.
Clause 13. The system of any proceeding or preceding clause wherein said lotion does not comprise alcohol.
Clause 14. A lotion for treating an irritant, said lotion comprising:
   zinc oxide;
   talcum;
   glycerin;
   water; and
   hydrocortisone.
Clause 15. The lotion of any proceeding or preceding clause comprising about 10 grams of zinc oxide, about 10 grams of talcum, about 7.5 cc of glycerin, and wherein water is added to a quantity sufficient to make 60 milliliters.

We claim:

1. A lotion for treating an irritant, said lotion comprising:
a protecting agent;
a moisture absorber;
a binding agent;
water;
an antibiotic agent.

2. The lotion of claim 1 wherein said protecting agent comprises zinc oxide, wherein said moisture absorber comprises talcum, wherein said binding agent comprises glycerin, and wherein said antibiotic agent comprises neomycin.

3. The lotion of claim 2 comprising about 10 grams of zinc oxide, about 10 grams of talcum, about 7.5 cc of glycerin, and neomycin.

4. The lotion of claim 3 wherein water is added to a quantity sufficient to make 60 milliliters.

5. The lotion of claim 4 comprising a density of about 12.64 g/mL.

6. The lotion of claim 4 wherein said lotion comprises about 5% to about 25% by weight zinc oxide, further wherein the lotion comprises between about 5% and about 25% by weight talcum,

7. The lotion of claim 1 wherein said lotion does not comprise alcohol.

8. A system for treating skin, said system comprising:
a dispenser for dispensing lotion, said dispenser having a body and a top,
wherein said body has a diameter greater than the diameter of said top;
a lotion located in said dispenser, said lotion comprising:
a protecting agent;
a moisture absorber;
a binding agent;
water;
an antibiotic agent.

9. The system of claim 8 wherein said protecting agent comprises zinc oxide, wherein said moisture absorber comprises talcum, wherein said binding agent comprises glycerin, and wherein said antibiotic agent comprises neomycin.

10. The system of claim 8 comprising about 10 grams of zinc oxide, about 10 grams of talcum, about 7.5 cc of glycerin, and neomycin.

11. The system of claim 8 wherein said dispenser comprises a dropper.

12. The system of claim 8 wherein said diameter of said body is more than 5 times the diameter at the top.

13. The system of claim 8 wherein said lotion does not comprise alcohol.

14. A lotion for treating an irritant, said lotion comprising:
zinc oxide;
talcum;
glycerin;
water; and
hydrocortisone.

15. The lotion of claim 14 comprising about 10 grams of zinc oxide, about 10 grams of talcum, about 7.5 cc of glycerin, and wherein water is added to a quantity sufficient to make 60 milliliters.
